(19) United States
(12) Patent Application Publication
(10) Pub. No.: US 2017/0112477 A1
(43) Pub. Date: Apr. 27, 2017

(54) TISSUE SAMPLE DEVICE AND METHODS

(71) Applicant: BOSTON SCIENTIFIC SCIMED, INC., Maple Grove, MN (US)

(72) Inventors: Christopher A. Benning, Hopkinton, MA (US); Andrew T. Brown, Shrewsbury, MA (US); John B. Golden, Norton, MA (US); Michael Eppihimer, Franklin, MA (US); Paul Smith, Smithfield, RI (US); Gerald Fredrickson, Westford, MA (US)

(21) Appl. No.: 15/297,622

(22) Filed: Oct. 19, 2016

Related U.S. Application Data
(60) Provisional application No. 62/244,578, filed on Oct. 21, 2015.

(51) Int. Cl. A61B 10/02; G01N 1/30

(52) U.S. Cl. CPC A61B 10/02; G01N 1/30; A61B 2010/0216; G01N 2001/305

(57) ABSTRACT

Medical devices and methods are disclosed in the present application. In one illustrative example a cell sample collection device may comprise an elongated shaft extending from a proximal end to a distal end and a tissue collection feature extending distal of the distal end of the elongated shaft. In at least some additional embodiments, at least a portion of the tissue collection feature may dissolve when placed in a fixation medium.

### CROSS-REFERENCE TO RELATED APPLICATIONS

This application claims priority under 35 U.S.C. §119 to U.S. Provisional Application Ser. No. 62/244,578, filed Oct. 21, 2015, the entirety of which is incorporated herein by reference.

### TECHNICAL FIELD

This disclosure relates to medical devices for tissue sample collection, and more particularly, to medical devices for tissue sample collection resulting in greater cellular or other tissue yield.

### BACKGROUND

Certain medical tests require sampling of cells from target areas of a subject's body. For instance, a screening test for detecting potentially pre-cancerous and cancerous tissues in a subject's body may include taking samples of tissue or cells from a target area of the subject's body. A tissue collection device may be used to collect cells or other tissues from the target area. Different tissue collection devices may differ in the amount of cells or other tissues they are able to collect or in the amount of cells or other tissues that are able to be successfully harvested from the tissue collection device. Devices that have higher cell or other tissue collection rates or higher harvestability of collected cells or other tissues may be beneficial.

### SUMMARY

This disclosure relates to medical devices for tissue sample collection, and more particularly, to medical devices for tissue sample collection resulting in greater cellular or other tissue yield. In a first illustrative embodiment, a cell sample collection device may comprise an elongated shaft extending from a proximal end to a distal end and a tissue collection feature extending distal of the distal end of the elongated shaft. In some embodiments, at least a portion of the tissue collection feature may dissolve when placed in a fixation medium.

Additionally, or alternatively, in any of the embodiments above with respect to the first illustrative embodiment, the portion of the tissue collection feature that dissolves when placed in the fixation medium may comprise a resin.

Additionally, or alternatively, in any of the embodiments above with respect to the first illustrative embodiment, the portion of the tissue collection feature that dissolves when placed in the fixation medium may comprise a pre-polymer.

Additionally, or alternatively, in any of the embodiments above with respect to the first illustrative embodiment, the portion of the tissue collection feature that dissolves when placed in the cell fixation medium may comprise polycarbonate.

Additionally, or alternatively, in any of the embodiments above with respect to the first illustrative embodiment, the portion of the tissue collection feature that dissolves when placed in the fixation medium may comprise acrylonitrile butadiene styrene (ABS).

Additionally, or alternatively, in any of the embodiments above with respect to the first illustrative embodiment, the portion of the tissue collection feature that dissolves when placed in the fixation medium may dissolve in under one hour when placed a fixation medium comprising a 1:1 solution of methanol and acetone.

Additionally, or alternatively, in any of the embodiments above with respect to the first illustrative embodiment, the portion of the tissue collection feature that dissolves when placed in the fixation medium may dissolve in under one hour when placed in a fixation medium comprising 100% liquid acetone.

Additionally, or alternatively, in any of the embodiments above with respect to the first illustrative embodiment, the portion of the tissue collection feature that dissolves when placed in 100% liquid xylene.

Additionally, or alternatively, in any of the embodiments above with respect to the first illustrative embodiment, the tissue collection feature comprises a brush, the brush comprising a center post and bristles connected to the center post.

Additionally, or alternatively, in any of the embodiments above with respect to the first illustrative embodiment, the portion of the tissue collection feature that dissolves when placed in the fixation medium comprises the bristles.

Additionally, or alternatively, in any of the embodiments above with respect to the first illustrative embodiment, the center post comprises a flat post, and wherein the bristles comprise a micropattern formed on the edges of the center post.

In a second illustrative embodiment, a cytology brush may comprise an elongated shaft including a proximal portion and a distal portion and a plurality of bristles disposed on the distal portion. In some embodiments, the bristles may be soluble in a fixation medium.

Additionally, or alternatively, in any of the embodiments above with respect to the second illustrative embodiment, the fixation medium comprises one of: a 1:1 solution of methanol and acetone, 100% liquid acetone, 100% methanol, 100% ethanol, formalin, and/or gluteraldeyde.

Additionally, or alternatively, in any of the embodiments above with respect to the second illustrative embodiment, the bristles comprise a resin.

Additionally, or alternatively, in any of the embodiments above with respect to the second illustrative embodiment, the bristles comprise at least one of: polycarbonate, ABS, and a polycarbonate and ABS blend.

Additionally, or alternatively, in any of the embodiments above with respect to the second illustrative embodiment, the bristles may dissolve in under one hour when placed in the fixation medium.

Additionally, or alternatively, in any of the embodiments above with respect to the second illustrative embodiment, the elongated shaft may be soluble in the fixation medium.

Additionally, or alternatively, in any of the embodiments above with respect to the second illustrative embodiment, the proximal portion of the elongated shaft may comprise a round shaft and wherein the distal portion of the elongated shaft comprises a flat shaft.

Additionally, or alternatively, in any of the embodiments above with respect to the second illustrative embodiment, the elongated shaft may comprise a twisted wire.

In a third illustrative embodiment, a method of preparing a cytology sample for analysis may comprise dissolving at least a portion of a cell sample collection device containing cells in a fixation medium.

In a fourth illustrative embodiment, a method of preparing a DNA or RNA sample for analysis may comprise dissolving at least a portion of a cell sample collection device containing cells in a buffer such as chloroform, phenol or a combination.

In a fifth illustrative embodiment, a method of preparing a tissue sample for histological analysis may comprise embedding a tissue sample collection in a histological media and dissolving at least a portion of the tissue collection device with Xylene.

Additionally, or alternatively, in any of the embodiments above with respect to the fifth illustrative embodiment, the histological media comprises one of paraffin or plastic.

Additionally, or alternatively, in any of the embodiments above with respect to the fifth illustrative embodiment, the method may further comprise sectioning the histological media with a microtome.

In a sixth illustrative embodiment, a tissue sample collection device may comprise an elongated shaft having a longitudinal axis including a proximal portion and a distal portion. The device may further comprise at least one ribbon spiraling around the longitudinal axis. In some embodiments, the ribbon may comprise a plurality of cuts extending in an outer edge of the ribbon extending toward the longitudinal to form bristles.

Additionally, or alternatively, in any of the embodiments above with respect to the sixth illustrative embodiment, at least a portion of the tissue sample collection device is soluble in a fixation medium.

Additionally, or alternatively, in any of the embodiments above with respect to the sixth illustrative embodiment, the bristles of the tissue sample collection device are soluble in a fixation medium.

The above summary of the present disclosure is not intended to describe each embodiment or every implementation of the present disclosure. Advantages and attainments, together with a more complete understanding of the disclosure, will become apparent and appreciated by referring to the following detailed description and claims taken in conjunction with the accompanying drawings.

### BRIEF DESCRIPTION OF THE DRAWINGS

The disclosure may be more completely understood in consideration of the following detailed description of various embodiments in connection with the accompanying drawings, in which:

FIG. 1 is a view of an example tissue collection device, in accordance with various embodiments of the present disclosure;

FIG. 2 is a close-up of an example distal end of the tissue collection device of FIG. 1 including bristles;

FIG. 3 is a close-up of another example distal end of the tissue collection device of FIG. 1 comprising a flat, twisted ribbon;

FIG. 4 is a close-up of another example distal end of the tissue collection device of FIG. 1 comprising two flat, twisting ribbons;

FIG. 5 is a close-up of another example distal end of the tissue collection device of FIG. 1 comprising a flat, twisted ribbon connected to a shaft;

FIG. 6 is a close-up of an example distal end of the tissue collection device of FIG. 1 including a frangible region;

FIGS. 7A-7B are example cross-sections of the frangible region of FIG. 6;

FIGS. 8A-8D are example atraumatic features that may be implemented on any of the tissue collection devices of FIGS. 1-5;

FIG. 9 is a flow diagram of an example method of collecting cells or other tissue using a tissue collection device such as the tissue collection devices of FIGS. 1-5;

FIGS. 10A-10E depict steps of the example method of FIG. 9;

FIG. 11 is a flow diagram of another example method of collecting cells or other tissue using a tissue collection device such as the tissue collection devices of FIGS. 1-5; and

FIGS. 12A-12C depict steps of the example method of FIG. 11.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended to be only exemplary. Selected features of any illustrative embodiments may be incorporated into any other described embodiments unless clearly stated to the contrary.

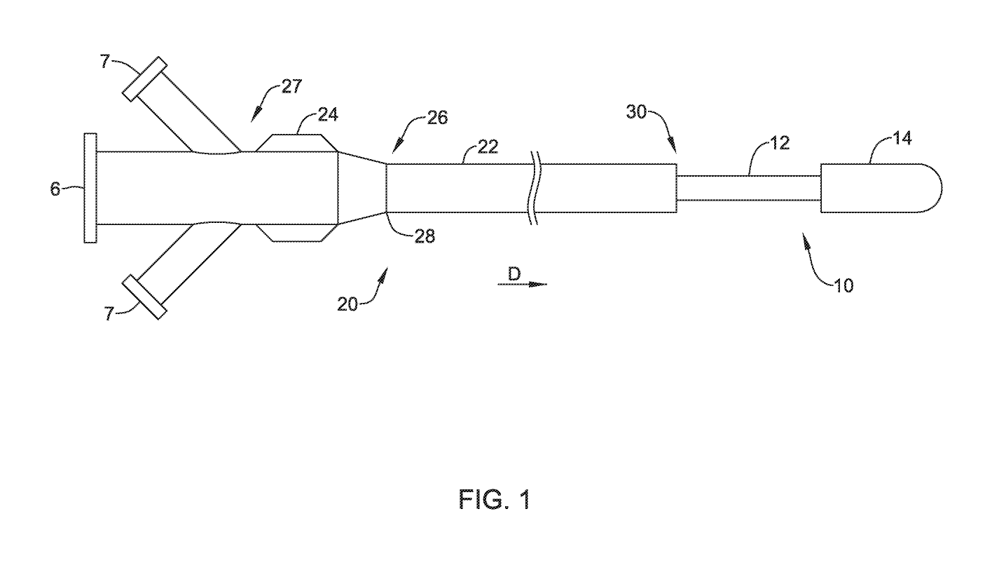

FIG. 1 shows an exemplary tissue collection device 10 in accordance with various embodiments of the present disclosure. In some cases, tissue collection device 10 may comprise shaft 12 and tissue collection feature 14. Tissue collection device 10 may be used to collect cells or other tissues from various locations within a patient's body. For example, tissue collection device 10 may be used to collect cells or other tissue from locations within the biliary tree. Tissue collection device 10 may be positioned at a desired location within the biliary tree, such as at a stricture or proximate a growth. Tissue collection feature 14 may then be maneuvered so as to collect cells or other tissue from the site.

In other cases, however, tissue collection device 10 may be used to collect cells or other tissues from locations within the esophagus, the gastro-intestinal tract, the vascular system, the cervix, or other locations within a patient's body. These collected cells or other tissues may then be used in one or more analyses. For instance, the cells or other tissues may be used in a histological analysis, flow cytometry analysis, immunohistochemstry analysis, molecular phenotyping analysis, nucleic acid detection, mRNA detection, or other types of analyses. These are just a few examples of how the collected cells or tissues may be used. In at least some of these embodiments, in order for the cells or tissues to be prepared for analysis, the cells or other tissues are placed in a fixation medium. The fixation medium may generally be configured to preserve the cells or other tissues from decay.

In general, tissue collection feature 14, or at least a portion of tissue collection feature 14, may be configured to be dissolvable within one or more commonly used fixation mediums or during one or more commonly used tissue processing techniques. For instance, tissue collection feature 14, or a portion of tissue collection feature 14, may be fully dissolvable within twenty minutes without agitation when placed in a 1:1 solution of methanol and acetone. However, in other embodiments, the ratio may range from between about 0.25:1 to about 1:0.25. In further embodiments, tissue collection feature 14, or a portion of tissue collection feature 14, may be additionally or alternatively fully dissolvable within twenty minutes when placed in 100% liquid acetone or when washed with 100% liquid xylene. In still further embodiments, tissue collection feature 14, or a portion of tissue collection feature 14, may be additionally or alternatively fully dissolvable within twenty minutes when placed in chloroform, phenol, triazol, or a mixture of chloroform, phenol, and/or triazol.

Some example materials that tissue collection feature 14 may be formed from include various pre-polymers and/or resins. Resins are generally solid or highly viscous substances, usually containing pre-polymers with reactive groups. Some more specific example materials include polycarbonate, acrylonitrile butadiene styrene (ABS), and blends of polycarbonate and ABS. Further specific example materials include Cycology™ Resin: HC1204HF, which is a blend of polycarbonate and ABS, Lexan™ Resin HP4, which is a medium flow polycarbonate, and Cycolac™ Resin HMG47MD, which is an ABS material. In other embodiments, tissue collection feature 14 may be formed from one or more polymers that have the desired dissolvability characteristics. Additionally, tissue collection feature 14 may be formed from any combination of these materials. It should be understood, however, that these are just a few example materials. Other materials which may dissolve in commonly used fixation mediums are also contemplated by this disclosure.

In further embodiments, the dissolvability of tissue collection feature 14 may differ. For instance, tissue collection feature 14 may be comprised of a material or materials that such that tissue collection feature 14, or at least a portion of tissue collection feature 14, dissolves in one or more common fixation mediums without agitation in under five hours, in under four hours, in under three hours, in under two hours, in under one hour, in under fifty minutes, in under forty minutes, in under thirty minutes, in under ten minutes, or any other suitable period of time.

The dissolvability of tissue collection feature 14 may increase the yield of the cellular material or other tissue collected on tissue collection feature 14 after sampling a target site within the patient. For instance, instead of attempting to separate the collected cells or other tissues from tissue collection feature 14, tissue collection feature 14 may be dissolved within the fixation medium, assuring a greater transfer of the collected cells or other tissues from tissue collection feature 14 to the fixation medium.

In some embodiments, tissue collection device 10 and/or tissue collection feature 14 may include one or more features that allow tissue collection device 10 and/or tissue collection device 10 to be viewable under one or more imaging modalities. For instance, in some embodiments, tissue collection device 10 and/or tissue collection feature 14 may be formed from a material having echogenic properties or may be formed to have structures that give tissue collection device 10 and/or tissue collection feature 14 echogenic properties. These echogenic properties may allow tissue collection device 10 and/or tissue collection feature 14 to be viewable under ultrasound. Additionally, or alternatively, tissue collection device 10 and/or tissue collection feature 14 may formed from radiopaque materials. For instance, radiopaque markers may be embedded along various portions of tissue collection device 10 and/or tissue collection feature 14. Alternatively, radiopaque materials may be mixed with the other materials used to form tissue collection device 10 and/or tissue collection feature 14 in order to allow tissue collection device 10 and/or tissue collection feature 14 to be viewed under fluoroscopy.

In different embodiments, tissue collection feature 14 may comprise different types of features. For instance, in some embodiments, tissue collection feature 14 may comprise bristles. However, in other embodiments, tissue collection feature 14 may be a flat, twisted ribbon with a micro-pattern formed on the edges of the ribbon. These and other embodiments will be described below with respect to other figures.

Shaft 12 of tissue collection device 10 may be connected to tissue collection feature 14. Alternatively, shaft 12 and tissue collection feature 14 may be formed as one, unitary component. In either embodiment, shaft 12 may be made from a variety of materials. For instance, shaft 12 may act as a pushwire for tissue collection feature 14, allowing tissue collection feature 14 to be advanced and retracted by advancing and retracting shaft 12. Accordingly, shaft 12 may be sufficiently rigid to transfer forces applied to shaft 12 at a point away from tissue collection feature 14 to tissue collection feature 14. However, where tissue collection device 10 needs to be maneuvered through one or more tortuous passageways of a patient to arrive at the target sampling site, shaft 12 may also need to be flexible enough to navigate those tortuous passageways.

In some embodiments, shaft 12 may be made from a metal or alloy. For instance, shaft 12 may be made from stainless steel, such as various varieties of stainless steel numbers 304, 316, 420, and 440, or other appropriate stainless steel varieties. Alternatively, shaft 12 may be formed of a super-elastic material such as the alloys of nickel and titanium, commonly known as Nitinol. While Nitinol is the most common super-elastic material, any of a variety of other super-elastic materials may be used for elongate member 11. Other alloys by chemical name include: CuAlNi, CuSn, CuZn, InTi, NiAl, FePt, MnCu, and FeMnSi. In addition to super-elastic materials, linear-elastic materials may be used. In general, linear-elastic materials are composed of the same alloys above. However, different material processing strategies are used to provide properties which has many of the important characteristics of a super-elastic material without some of the difficulties related to machining, specifically grinding. As one example, shaft 12 may preferably be formed of a linear-elastic alloy of nickel-titanium.

In still further embodiments, shaft 12 may be formed from any of the materials discussed above with respect to tissue collection feature 14. For instance, shaft 12 may be formed from the same material as tissue collection feature 14. Although, in other embodiments, shaft 12 may be formed from one of materials listed above for tissue collection feature 14, but tissue collection feature 14 may be formed from a different one of the listed materials. In these embodiments, shaft 12 may have similar dissolvability properties as tissue collection feature 14.

In still other embodiments, shaft 12 may comprise a proximal portion and a distal portion formed from different materials. For instance, in some embodiments, the proximal portion of shaft 12 may be made from a metal, such as any of the ones listed above. The distal portion of shaft 12 may be formed from one or more of the materials listed above with respect to tissue collection feature 14 and may be attached to the proximal portion of shaft 12. In these embodiments, tissue collection feature 14 may comprise features connected to, or formed on, the distal portion of shaft 12. In other embodiments, shaft 12 may be formed from a single material, and tissue collection feature 14 may comprise a center shaft and one or more features connected to, or formed on, the center shaft. The center shaft of tissue collection feature 14 may then be connected to the distal end of shaft 12. These different embodiments will be described in more detail below with respect to other figures.

In some embodiments, tissue collection device 10 may be used as a stand-alone device. For instance, tissue collection device 10 may be maneuvered, using shaft 12, into a passageway of a patient proximate a site of interest. Tissue collection feature 14 may then collect sample cells or other tissues from the site of interest, and tissue collection device 10 may be withdrawn from the patient.

However, in other embodiments, tissue collection device 10 may be used with a catheter or endoscope, as represented by device 20 in FIG. 1. Device 20 may be a guide or diagnostic catheter, or another medical device such as an endoscope or hysteroscope, and may have a length and an outside diameter appropriate for its desired use. For example, where device 20 is adapted as a guide catheter, device 20 may have a length of about 20-250 cm and an outside diameter of approximately 1-10 French, depending upon the desired application. In some cases, device 20 may be a microcatheter that is adapted and/or configured for use within small anatomies of the patient. For example, device 10 may be used to navigate to targets sites located in tortuous and narrow vessels such as, for example, to sites within the neurovascular system, certain sites within the coronary vascular system, or to sites within the peripheral vascular system such as superficial femoral, popliteal, or renal arteries. In some cases, the target site is a neurovascular site and may be located within a patient's brain, which is accessible only via a tortuous vascular path. In still other embodiments, device 20 may be an endoscope or hysteroscope and may be sized according to common endoscope or hysteroscope sizes.

As shown in FIG. 1, device 20 can include elongate shaft 22. Elongate shaft 22 may generally extend from proximal portion 26 and proximal end 28 in distal direction D toward distal portion 30. Although depicted as having a generally circular shape, it will be understood that elongate shaft 22 can have other shapes or combinations of shapes without departing from the scope of the disclosure. For example, the shape of the generally tubular elongate shaft 22 may be oval, rectangular, square, triangular, polygonal, and the like, or any other suitable shape, depending upon the desired characteristics.

In some cases, manifold 24 may be connected to proximal end 28 of elongate shaft 22. The manifold may include hub 27 and/or other structures to facilitate connection to other medical devices (e.g., syringe, stopcocks, Y-adapter, etc.) and to provide access to one or more lumens defined within elongate shaft 22. In some cases, hub 27 may include ports 6, 7, and 8, which provide individual access to one or more lumens extending through at least a portion of device 20. Some example lumens that may extend through device 20 may include at least one guidewire lumen, where device 20 is an over-the-wire device, and a working channel sized to cooperate with tissue collection device 10. The lumens that do extend through device 20 may terminate at or near distal portion 30 of elongate shaft 22. However, in other cases, hub 27 may have a single port, two ports, or any other number of ports. Manifold 24 may also include a strain relief portion adjacent proximal end 28 of elongate shaft 22.

Where device 20 is a catheter or other flexible medical device configured to navigate tortuous body vessels, device 20, or at least elongate shaft 22, may be made from a polymer (some examples of which are disclosed below), a metal-polymer composite, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Where device 20 is a device such as an endoscope or hysteroscope, device 20 may be made from a suitable metal. For example, device 20, or at least shaft 22 of device 20, may be made from a suitable medical grade stainless steel.

Accordingly, where tissue collection device 10 is configured to cooperate with device 20, tissue collection device 10 may be sized to fit within a lumen of device 20. For instance, shaft 12 and tissue collection feature 14 may have diameters smaller than a lumen or channel of device 20 to allow insertion of tissue collection device 10 into the lumen or channel. As described, device 20 may have a variety of different sizes depending on the location of the target sampling site. Accordingly, tissue collection device 10 may also have a variety of sizes depending on the location of the target sampling site.

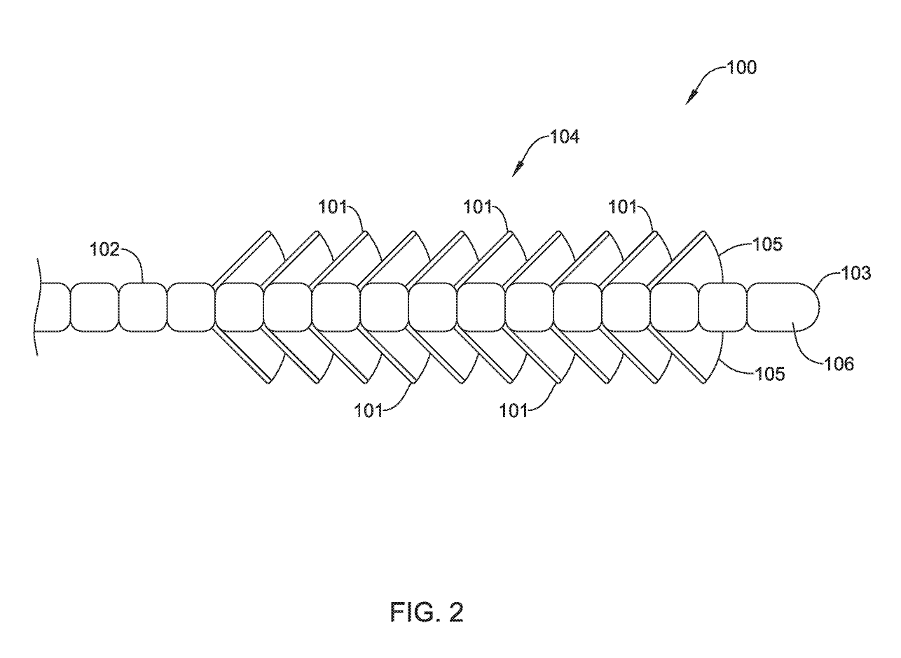

FIG. 2 is a close-up of the distal end of example tissue collection device 100. Tissue collection device 100 may include shaft 102 and tissue collection feature 104. In the embodiment of FIG. 2, shaft 102 may be a twisted or braided structure and may extend from a proximal end (not shown) to distal end 103. For instance, shaft 102 may be comprises of two or more elongate components that are twisted or braided together to form shaft 102. Shaft 102 may be formed from any of the materials discussed above with respect to shaft 12.

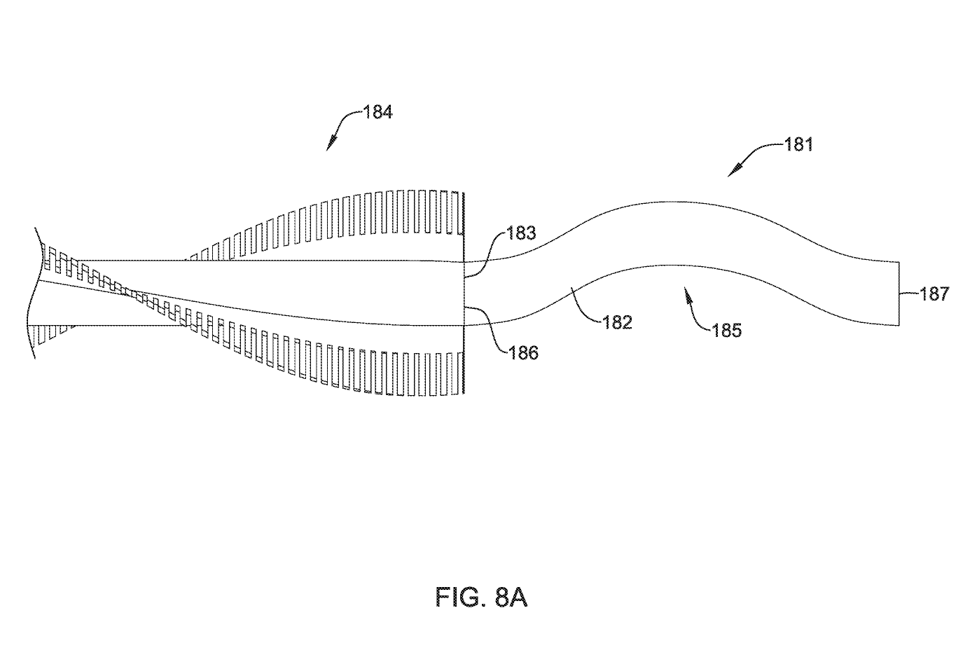

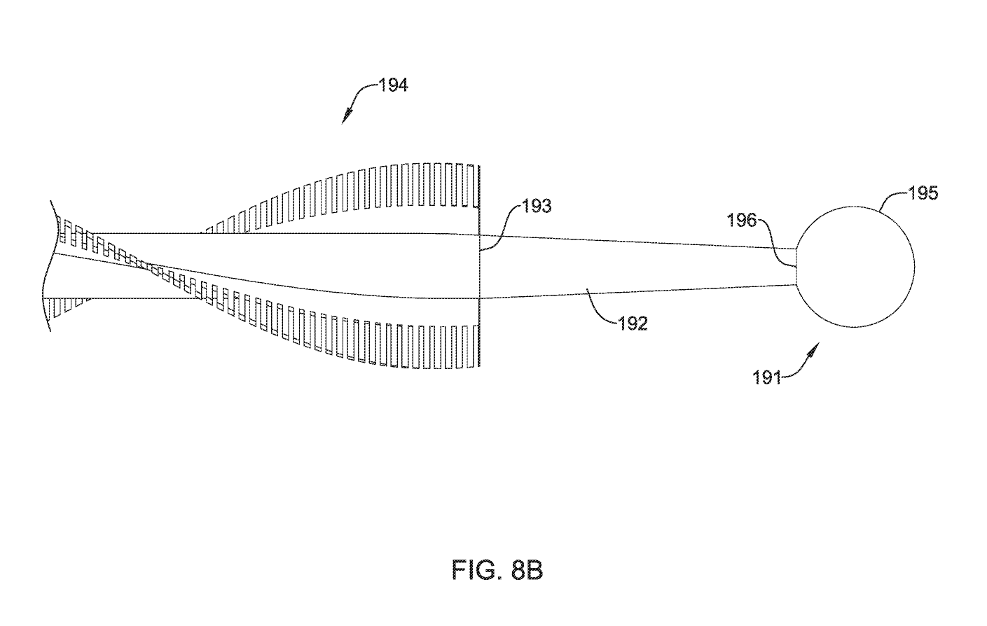

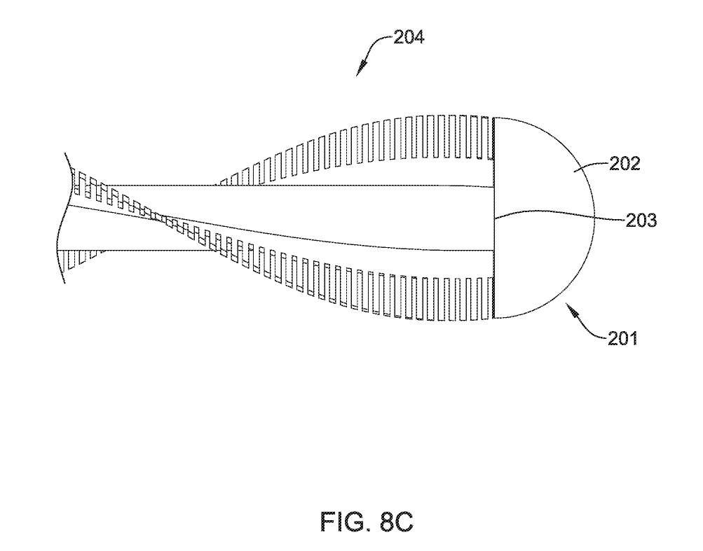

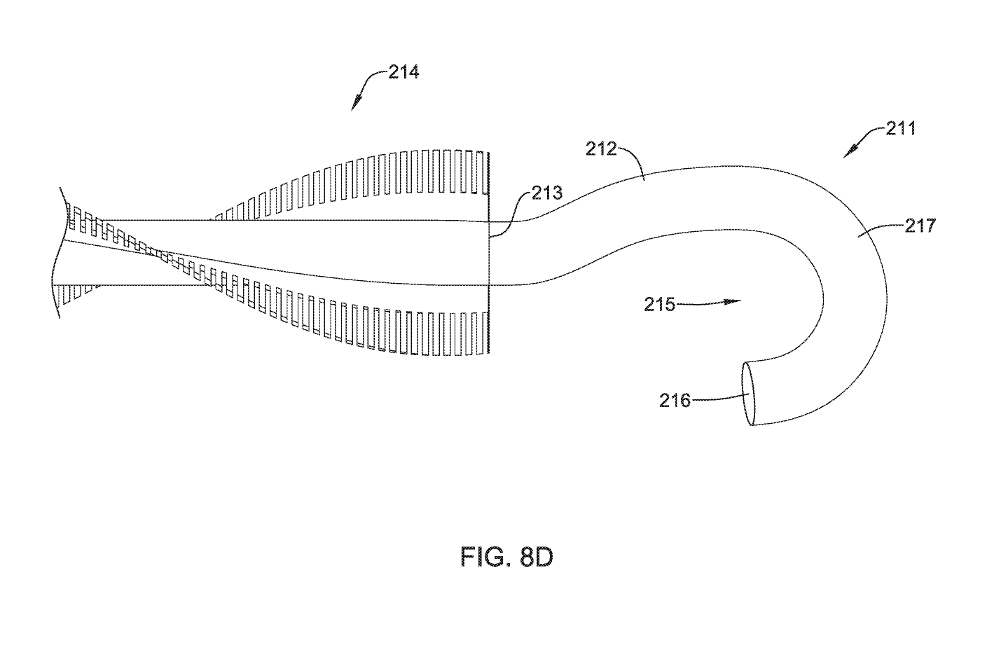

In some embodiments, shaft 102 may terminate in atraumatic tip 106. Generally, atraumatic tip 106 may be configured to not catch on tissue as tissue collection device 100 is advanced inside a patient. Further embodiments of atraumatic tips that may be used with tissue collection device 100 are described with respect to FIGS. 8A-8D.

FIGS. 2-5 depict example structures of tissue collection devices or features that may be used to collect cells or other tissues in accordance with the present disclosure. In the embodiment of FIG. 2, tissue collection feature 104 comprises bristles 101 disposed on a distal portion of shaft 102. Bristles 101 may be formed of a material such as those described with respect to tissue collection feature 14 of FIG. 1 such that bristles 101 may dissolve in commonly used fixation mediums.

In the example of FIG. 2, bristles 101 are depicted as extending outward from shaft 102 on two sides of shaft 102. However, in other embodiments, bristles 101 may extend outward from shaft 102 in all directions so that shaft 102 is covered with bristles over all 360 degrees around shaft 12. Additionally, bristles 101 may form an angle 105 with respect to shaft 102. In some embodiments, angle 105 may be between about 45 degrees and about 135 degrees. In more specific embodiments, angle 105 may be about 45 degrees, about 60 degrees, about 90 degrees, about 120 degrees, about 135 degrees, or any other suitable value. However, it should be understood that these are only a few examples of how bristles 101 may be oriented with respect to shaft 102. In general, bristles 101 may have any geometry with respect to shaft 102 and may cover a length of shaft 102 between about 2 cm to about 20 cm.

Bristles 101 may be attached to shaft 102 in any suitable manner. For instance, bristles 101 may connected to shaft 102 using a suitable adhesive material. Alternatively, bristles 101 may be positioned between the two or more elongate components of shaft 102 before they are twisted or braided together. With bristles 101 disposed therebetween, the twisting or braiding of the elongate components of shaft 102 then secures bristles 101 between the elongate members of shaft 102. However, it should be understood that these are only a few examples of how bristles 101 may be attached or secured to shaft 102.

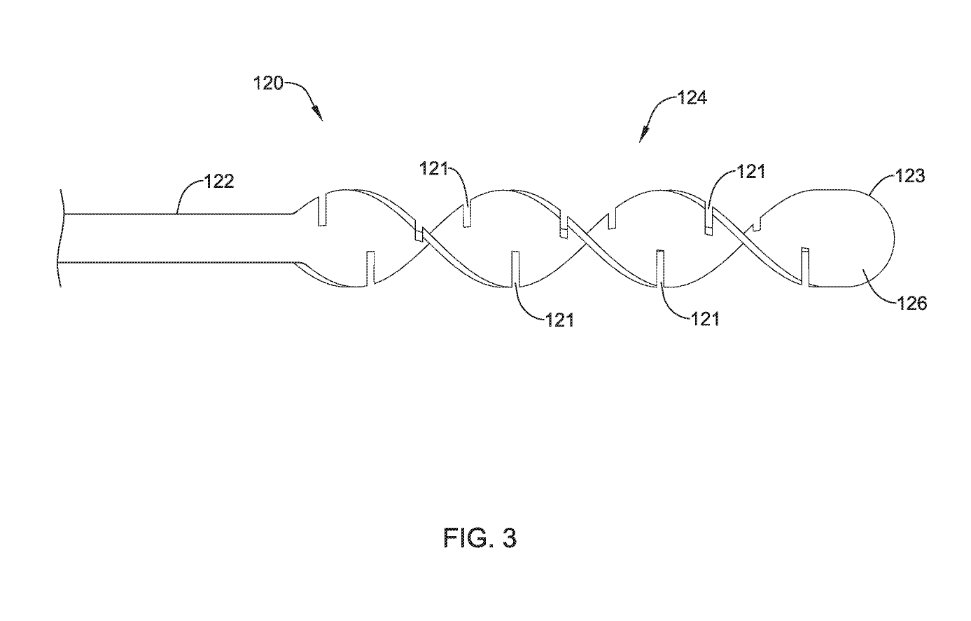

FIG. 3 depicts another example embodiment of a tissue collection device, tissue collection device 120. Tissue collection device 120 may comprise shaft 122 along with tissue collection feature 124. In the example of FIG. 3, shaft 122 and tissue collection feature 124 may be formed as a signal piece. For instance, as can be seen, shaft 122 may generally be a round structure and may transition into a flat, twisted ribbon forming tissue collection feature 124. Tissue collection feature 124 may have a helical twist so as to provide edges in all directions for contacting tissue when tissue collection feature 124 is disposed proximate a target sampling site.

In some embodiments, tissue collection feature 124 may include cut-outs 121 which may provide an increase in the collection of cells or other tissues by tissue collection feature 124. For instance, cut-outs 121 may provide additional locations for cells or other tissue to collect as tissue collection feature 124 is maneuvered at a target sampling site.

Tissue collection device 120 device may include a rounded distal end 123. Rounded distal end 123 may provide an atraumatic end to tissue collection device 120 in order to not damage tissue as tissue collection feature 124 is maneuvered at a target sampling site. Other example atraumatic tips that other embodiments of tissue collection device 120 may include are depicted in FIGS. 8A-8D.

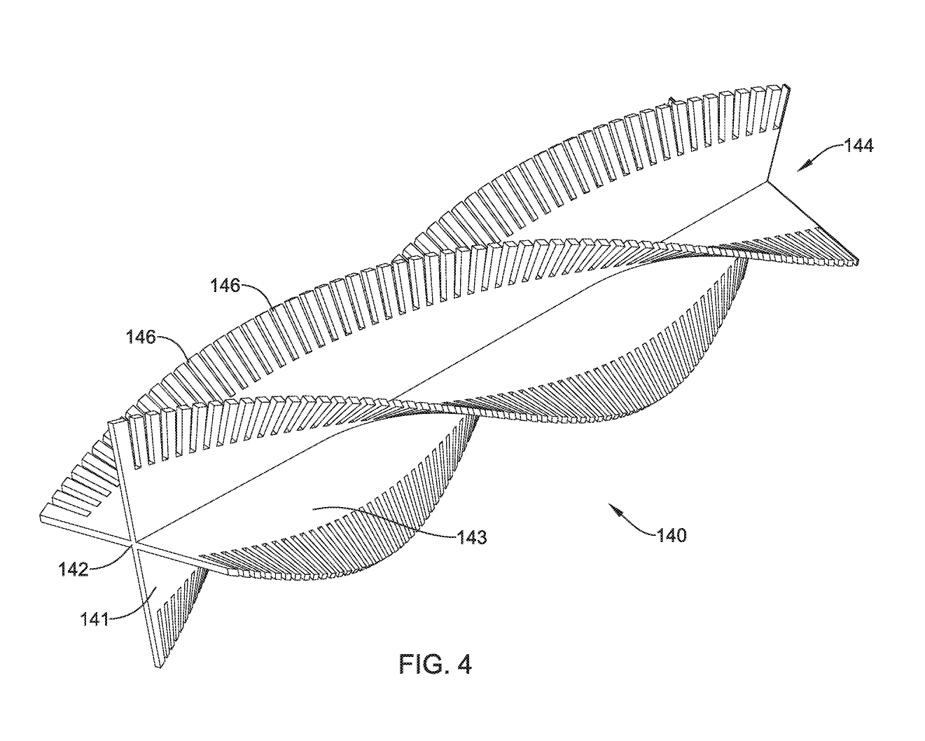

FIG. 4 depicts another example of a tissue collection feature, tissue collection feature 140. In the embodiment of FIG. 4, tissue collection feature 140 may comprise helically twisted ribbons 141 and 143. Ribbons 141, 143 may be generally oriented opposite to one another. In other embodiments, however, tissue collection feature 140 may only include a single ribbon.

In some embodiments, tissue collection feature 140 may comprise tissue collection structures 146 disposed on the edges of ribbons 141, 143. In some embodiments, tissue collection structures 146 may be small cut-outs similar to cut-outs 121 of FIG. 3 forming individual fingers. In other embodiments, however, tissue collection structures 146 may be striations carved into ribbons 141, 143 without cutting all the way through ribbons 141, 143.

In still other embodiments, tissue collection structures 146 may be micropatterns formed on the edges of ribbons 141, 143. The micropatterns may include a plurality of microstructures, for instance protrusions, extending radially outward from the outer edges of ribbons 141, 143. The microstructures may be cylindrical in some embodiments, with a circular cross-sectional shape. However, it should be understood that the microstructures may have any other suitable shape. For example, the microstructures may have a rectangular, oval, elliptical, oblong, spherical, triangular, hexagonal, or irregular cross-sectional shape.

The microstructures may further be spaced from each other to define a gap or space between adjacent microstructures. Cells or other tissues, or portions thereof, may enter these gaps between the microstructures and may be held therein by engagement with the microstructures on each side of the gaps. In some embodiments, the gaps between the microstructures may have a length in the range of 10-20 micrometers, and the microstructures may have a width of between 10 and 20 micrometers. The dimensions of the gap between the microstructures and the microstructures themselves may vary, however, based on the type of target cells or other tissues to capture. Of course, it should be understood that these are only a few examples of micropatterns that may be formed on ribbons 141, 143.

Proximal end 142 of tissue collection feature 140 may be attached to a shaft, such as shaft 12 of FIG. 1, in order to provide tissue collection feature 140 with support and to allow a user to maneuver tissue collection feature 140 remote from tissue collection feature 140. Additionally, tissue collection feature 140 may include an atraumatic tip connected to distal end 144. Example atraumatic tips that may be used with tissue collection feature 140 are described with respect to FIGS. 8A-8D.

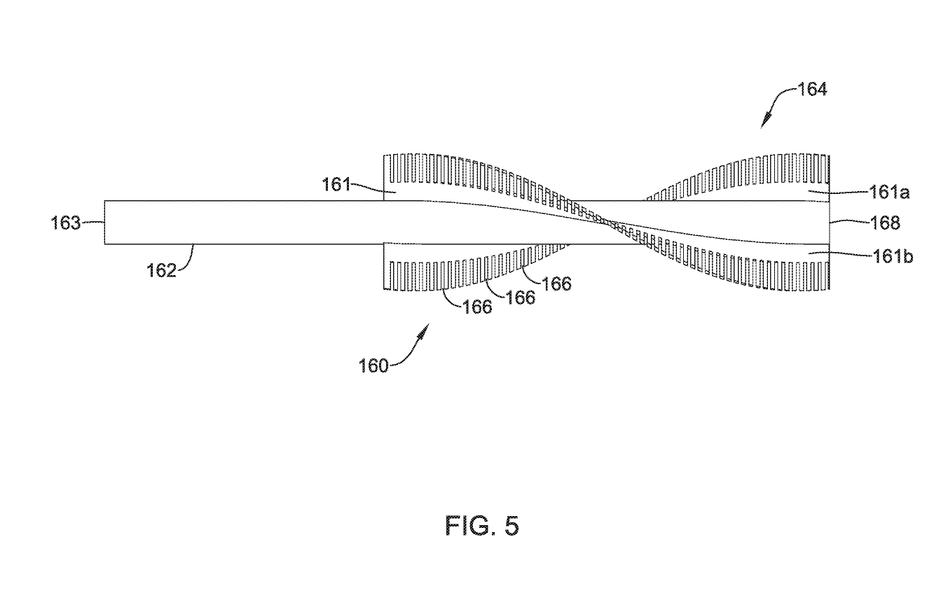

FIG. 5 depicts yet another example tissue collection device, tissue collection device 160. In the embodiment of FIG. 5, tissue collection device 160 may include shaft 162 and tissue collection feature 164. Tissue collection feature 164 may be comprised of twisting ribbon 161 that is formed directly on, or connected to, shaft 162. In other embodiments, ribbon 161 may comprise two portions, 161a and 161b, either formed directly on, or connected to, opposite sides of shaft 162. Additionally, in other embodiments, tissue collection feature 164 may include another twisting ribbon formed directly on, or connected to, shaft 162 oriented in an opposing direction to ribbon 161.

In some embodiments, tissue collection feature 164 may further include tissue collection structures 166. Tissue collection structures 166 may be similar to any of the tissue collection structures described with respect to tissue collection structures 146 of FIG. 4.

Additionally, in some embodiments of FIG. 5, shaft 162 may be formed from similar materials to tissue collection feature 164 and also has desired dissolvability characteristics. In at least some of these embodiments, shaft 162 is attached, at proximal end 163 to another shaft (not shown), which may be made from a different material than shaft 162, such as a metallic element or alloy.

Tissue collection device 160 may include an atraumatic tip connected to distal end 168. Example atraumatic tips that may be used with tissue collection device 160 are described with respect to FIGS. 8A-8D.

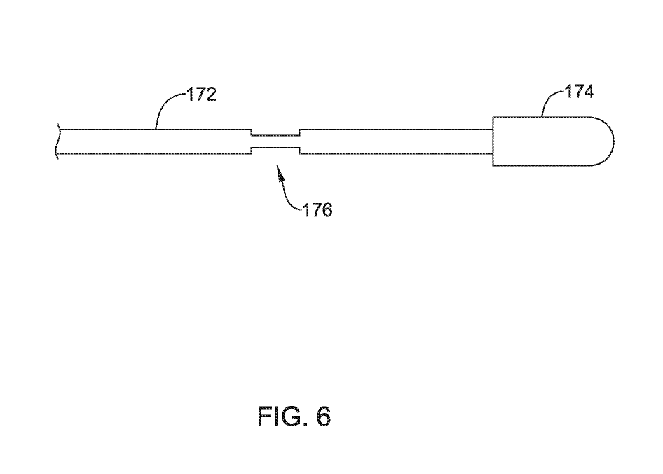

FIG. 6 depicts a close-up of a distal portion of shaft 172 including tissue collection feature 174. Shaft 172 includes frangible portion 176 disposed proximal of tissue collection feature 174. In general, frangible portion 176 may comprise an area of structural weakness of shaft 172 such that shaft 172 is more likely to break at frangible portion 176 when subjected to stresses. Shaft 172 and frangible portion 176 are depicted in FIG. 6 in a general manner, and it should be understood that any of the tissue collection devices or features of the present disclosure may include a frangible portion as described with respect to FIG. 6.

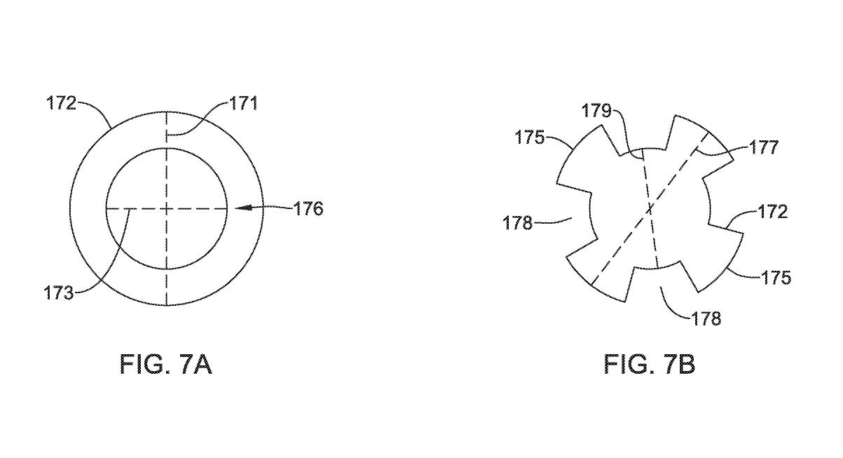

In some embodiments, frangible portion 176 may comprise an area of reduced cross-section, as shown in different embodiments in FIGS. 7A-7B. For instance, the outer diameter of shaft 172 in the area of frangible portion 176 may be reduced around the entirety of shaft 172, as shown in FIG. 7A. Diameter 171 depicts the outer diameter of shaft 172 in areas outside of frangible portion 176. Diameter 173 depicts the outer diameter of shaft 172 in areas within frangible portion 176.

FIG. 7B depicts an alternative embodiment of frangible portion 176. FIG. 7B depicts an example cross-sectional area of frangible portion 176 of shaft 172. In the embodiment of FIG. 7B, shaft 172, in frangible portion 176, may have portions 178 where outer diameter 179 of shaft 172 is less than the outer diameter of shaft 172 (not shown) outside of frangible portion 176. However, in the embodiment of FIG. 7B, shaft 172 may also have portions 175 where outer diameter 177 is the same as the outer diameter of shaft 172 (not shown) outside of frangible portion 176.

In some embodiments, where shaft 172 is an extruded body, frangible portion 176 may be formed during the extrusion process by drawing down the diameter of the extruded material. Alternatively, frangible portion 176 may be formed after formation of shaft 172 by removing material in the area of frangible portion 176, such as by cutting, burning, or melting material off of shaft 172. In still other embodiments, frangible portion 176 may not represent an area of reduced cross-section or smaller outer diameter. Rather, frangible portion 176 may be a weakened area relative to the rest of shaft 172. For instance, frangible portion 176 may be formed of a different, weaker material or may be mechanically weakened after formation of shaft 172.

FIGS. 8A-8D all depict example atraumatic tips that may be used in any of the tissue collection devices or features of the present disclosure. FIG. 8A depicts atraumatic feature 181, which may generally comprise shaft 182 connected to distal end 183 of tissue collection feature 184 and having bend 185 disposed part way between proximal end 186 and distal end 187 of shaft 182.

FIG. 8B depicts atraumatic feature 191 which may generally comprise shaft 192 connected to distal end 193 of tissue collection feature 194 and having bead 195 disposed on distal end 196 of shaft 192.

FIG. 8C depicts atraumatic feature 201 which may generally comprise rounded top 202 connected to distal end 203 of tissue collection feature 204.

FIG. 8D depicts atraumatic feature 211, which may generally comprise shaft 212 connected to distal end 213 of tissue collection feature 214. Shaft 212 may include bend 215 proximate distal end 216 of shaft 212 forming hook 217.

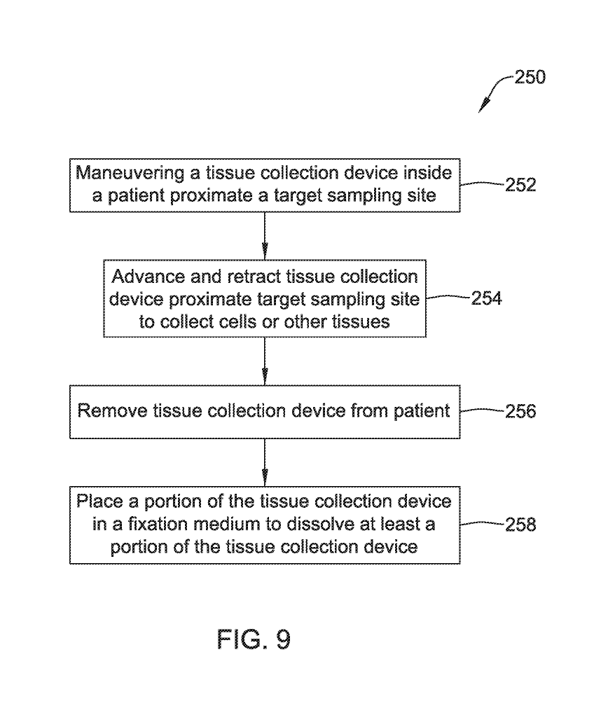

FIG. 9 depicts flow diagram 250 illustrating an example method of collecting cells or other tissue using a device such as any of the devices described with respect to FIGS. 1-5. The method may begin with inserting a tissue collection device, such as any of the devices described with respect to FIGS. 1-5, into a patient and maneuvering the device proximate a target sampling site, as at 252. In some embodiments, the target sampling site may be located within the biliary tree of the patient. However, in other embodiments, the tissue collection device may be used at other locations within the body, such as within the esophagus, the gastro-intestinal tract, the vascular system, the cervix, and other locations.

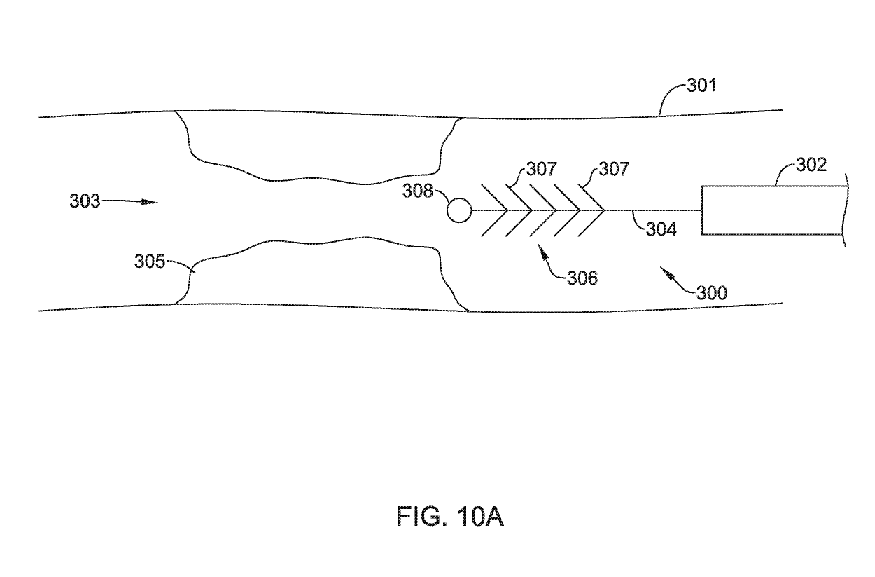

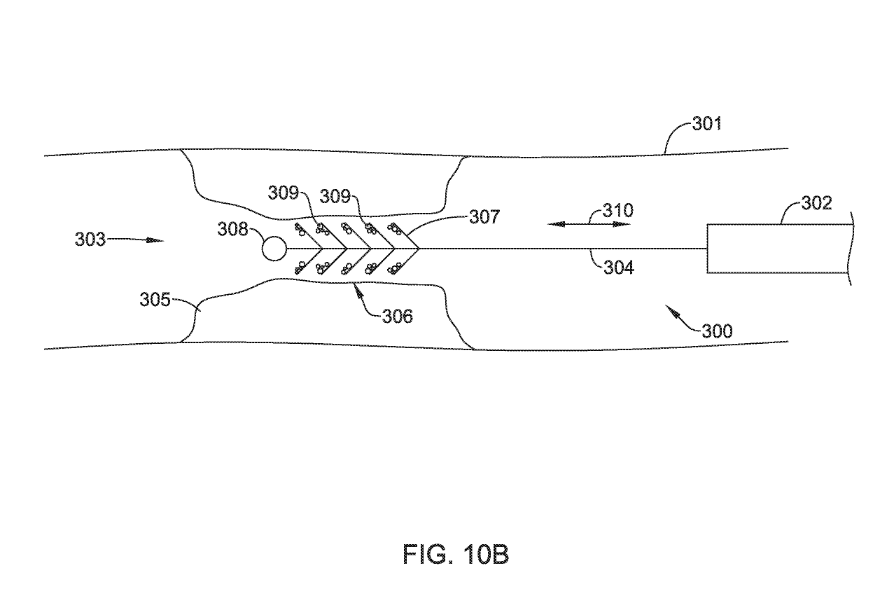

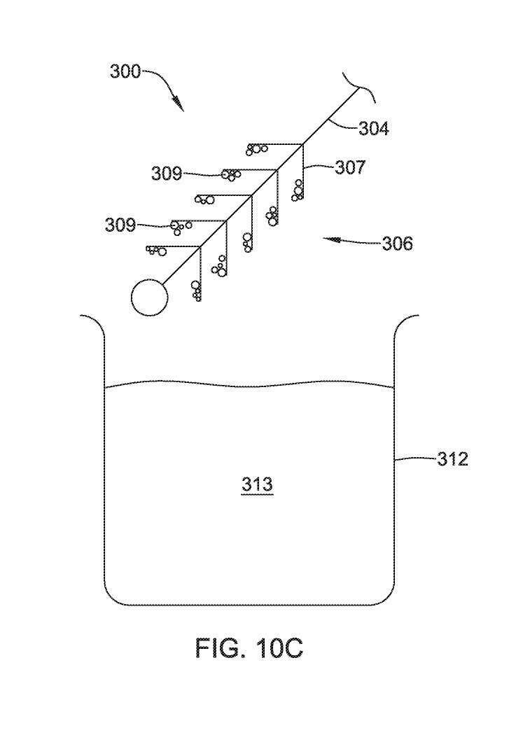

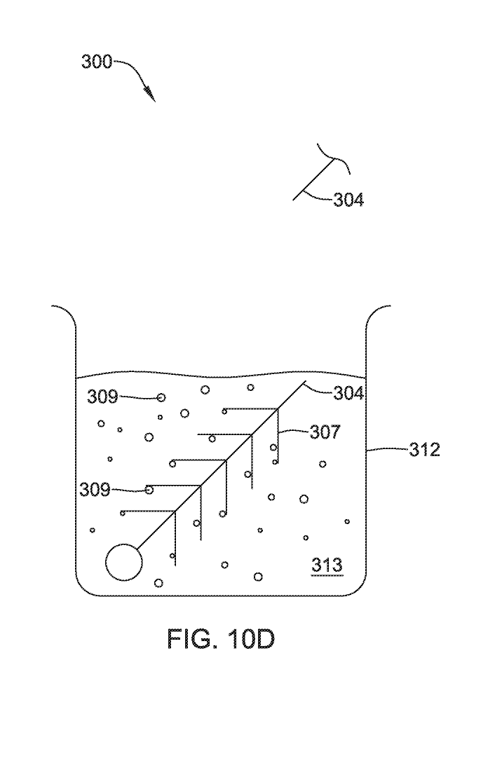

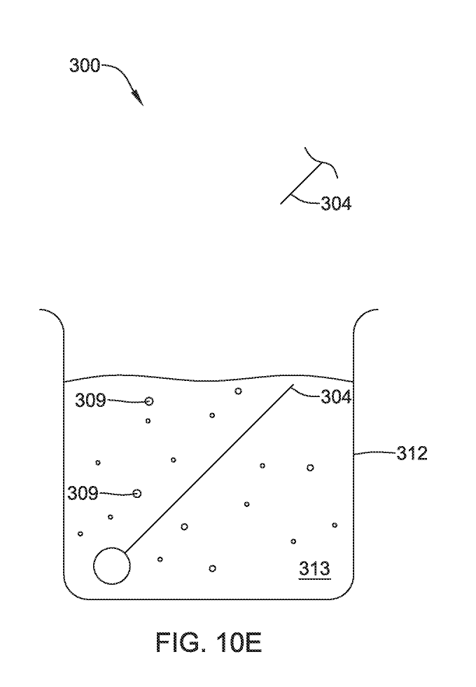

FIG. 10A depicts example tissue collection device 300 located at constriction 303 within biliary tract 301. Constriction 303 may be caused by growth 305. In order to determine the cause of growth 305, a user may sample growth 305 using tissue collection device 300 to collect cells or other tissue of growth 305 for analysis.

Tissue collection device may include shaft 304, tissue collection feature 306, and atraumatic tip feature 308. In the example of FIG. 10A, tissue collection feature 306 may comprise bristles 307. However, it should be understood that bristles 307 are just one example of tissue collection feature 306. In other embodiments, tissue collection device 300 and/or tissue collection feature 306 may be similar to any of the embodiments of the present disclosure. Additionally, atraumatic tip feature 308 is shown as a bead. However, in other embodiments, atraumatic tip feature 308 may be shaped differently, for instance in accordance with any of the other atraumatic tip embodiments of the present disclosure.

In some embodiments, tissue collection device may be used in conjunction with sheath 302. Sheath 302 may be a part of a medical device, such as any described with respect to device 20 of FIG. 1. In these embodiments, tissue collection device 300 may be transported through the patient to constriction 303 inside of sheath 302. Once sheath 302 is disposed proximate constriction site 303, sheath 302 may be retracted and/or tissue collection device 300 may be advanced to expose tissue collection feature 306 distal of the distal end of sheath 302. Of course, in other embodiments, tissue collection device 300 may be used without sheath 302.

Once tissue collection device 300 has been positioned proximate the target sampling site, such as constriction 303, tissue collection device 300 may be advanced and retracted proximate the target sampling site to collect cells or other tissues, as at 254. FIG. 10B depicts an example of this process. For instance, tissue collection device 300 may be advanced and retracted through constriction 303 in the direction of arrows 310. This movement may cause bristles 307 to contact growth 305 and to collect cells or other tissues 309 on bristles 307. Next, tissue collection device 300 may be removed from the patient, as at 256. FIG. 10C depicts tissue collection device 300 disposed outside of the patient and proximate container 312 containing fixation medium 313. Fixation medium 313 may comprise any standard fixation medium for fixing cells or other tissues including 1:1 solution of methanol and acetone, 100% liquid acetone, 100% methanol, and/or 100% ethanol. In still other embodiments, fixation medium 313 may comprise 100% liquid xylene, chloroform, and/or phenol, or a mixture of chloroform and phenol. In these last embodiments, the fixation medium may not cause a fixing of the cells disposed on tissue collection device 300, but may be a part of the tissue preparation process.

A portion of tissue collection device 300 containing tissue collection feature 306 may then be placed within the fixation medium to dissolve at least a portion of the tissue collection device, as at 258. In the example of FIG. 10D, a portion of tissue collection device 300, such as the portion including tissue collection feature 306, may be removed from the rest of tissue collection device 300 and placed within container 312. For instance, tissue collection device 300 may include a frangible portion that allows for easy separation of tissue collection feature 306 from tissue collection device 300. However, this is not necessary in all embodiments. In other examples, tissue collection feature 306 may be placed within fixation medium 313 without being separated from the rest of tissue collection device 300.

FIG. 10D depicts a portion tissue collection device 300 including tissue collection feature 306 disposed in container 312 containing fixation medium 313. At this point, cells or other tissues 309 may separate from tissue collection feature 306 and dissolve in fixation medium 313, as shown in FIG. 10D.

FIG. 10E depicts tissue collection feature 306 dissolved in fixation medium 313, allowing further transfer of cells or other tissues 309 into fixation medium 313. Although FIG. 10E depicts shaft 304 as not dissolved in fixation medium 313, in other embodiments, shaft 304 may be formed of a similar material as tissue collection feature 306. Accordingly, in these embodiments, shaft 304 may also dissolve in fixation medium 313.

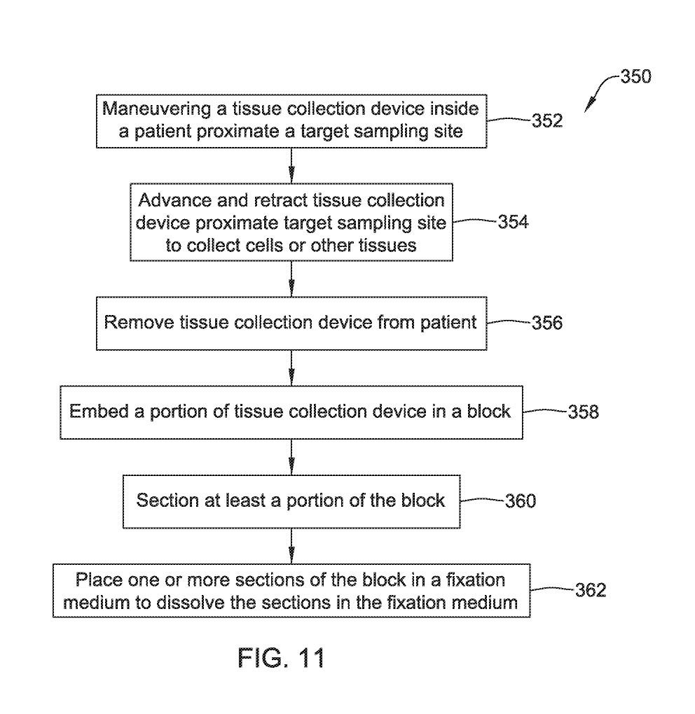

FIG. 11 depicts flow diagram 350 of an alternative method of collecting cells or other tissue and processing those cells or other tissues for analysis using a device such as any of the devices described with respect to FIGS. 1-5.

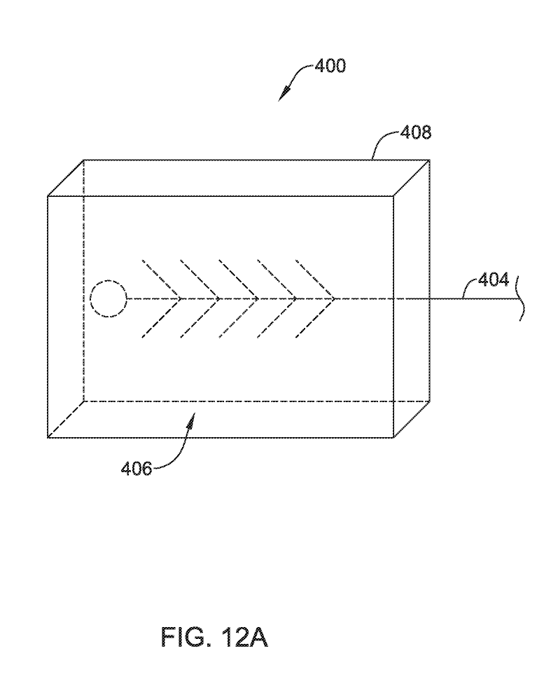

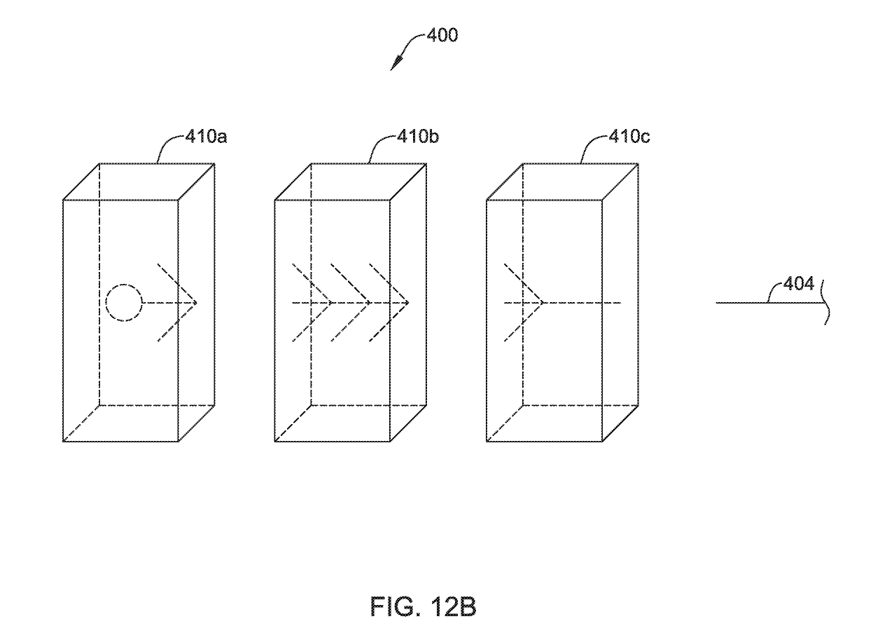

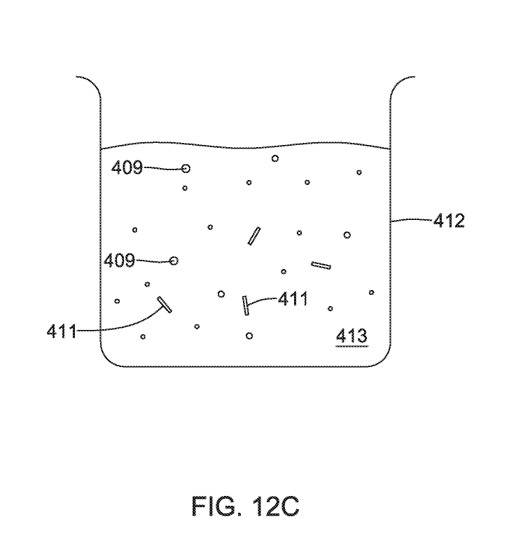

Steps 352-356 may be the same as steps 252-256 of FIG. 9. Once the tissue collection device has been removed from the patient, at least a portion of the tissue collection device may be embedded within a block, as at 358. For instance, a portion of the tissue collection device may be embedded within paraffin, plastic, or thrombin-plasma to form the block. FIG. 12A depicts an example tissue collection device 400, comprising shaft 404 and tissue collection feature 406, embedded within block 408. As cells or other tissues may have collected on tissue collection device 400 during the sampling process of steps 352-356, the cells or other tissues may also be embedded within block 408.

Next, the block may be sectioned into thin slices, as at 360. FIG. 12B depicts tissue collection device 400 sectioned into three sections, sections 410a, 410b, and 410c. However, this is just an example. In general, tissue collection device 400 may be sectioned into as many sections as desired. In at least some embodiments, sections 410a, 410b, and 410c may be between about 1 micrometer and about 50 micrometers thick. Block 408 may be sectioned, for instance, using a microtome. Accordingly, tissue collection device 400, or at least the portion of tissue collection device 400 embedded in block 408, may be formed of a material that may be cut by a microtome without damaging the microtome, such as a plastic or any of the materials listed with respect to tissue collection feature 14 of FIG. 1.

Finally, one or more of sections 410a, 410b, and 410c may be placed in a liquid medium to dissolve sections 410a, 410b, and 410c, including any cells or other tissues embedded within sections 410a, 410b, and 410c, as at 362. FIG. 12C depicts partially dissolved sections 410a, 410b, and 410c disposed within container 412 containing liquid medium 413. FIG. 12C depicts cells or other tissue 409 dissolved within liquid medium 413 along with portions of tissue collection device 411 partially dissolved. With more time in fixation medium 413, portions 411 may dissolve completely.

In some variations on method 350, instead of dissolving sections 410a, 410b, and 410c in a container, sections 410a, 410b, and 410c may be placed onto one or more slides. A fixation medium/solvent may then be applied to sections 410a, 410b, and 410c on the slides to dissolve the material of block of sections 410a-c and the portions of tissue collection device 400 embedded within sections 410a-c. Alternatively, the slides may be placed into the fixation medium/solvent instead of the fixation medium/solvent being applied to the slides. This process may leave the cells or other tissues on the slide and/or may fix the cells or other tissues.

In at least some embodiments, the method of FIG. 11 may be useful when tissue collection device 400 is made from plastic or where tissue collection feature 406 has a ribbon-like structure, as described with respect FIGS. 3-5. In these embodiments, sectioning tissue collection device 400 into small sections may improve the time in which tissue collection device 400 fully dissolves in fixation medium 413. Where tissue collection device 400 is formed from a plastic, a fixation medium/solvent of 100% acetone may be particularly useful.

After the steps of either method 250 or method 350, the prepared cells or other tissues (e.g. dissolved in a fixation medium/solvent or disposed on slides) may be ready for analysis.

Those skilled in the art will recognize that the present disclosure may be manifested in a variety of forms other than the specific embodiments described and contemplated herein. Specifically, the various features described with respect to the various embodiments and figures should not be construed to be applicable to only those embodiments and/or figures. Rather, each described feature may be combined with any other feature in various contemplated embodiments, either with or without any of the other features described in conjunction with those features. Accordingly, departure in form and detail may be made without departing from the scope of the present disclosure as described in the appended claims.

What is claimed:

1. A tissue sample collection device comprising:
an elongated shaft extending from a proximal end to a distal end; and
a tissue collection feature extending distal of the distal end of the elongated shaft,
wherein at least a portion of the tissue collection feature dissolves when placed in a fixation medium.

2. The tissue sample collection device of claim 1, wherein the portion of the tissue collection feature that dissolves when placed in the fixation medium comprises a resin.

3. The tissue sample collection device of claim 1, wherein the portion of the tissue collection feature that dissolves when placed in the fixation medium comprises a pre-polymer.

4. The tissue sample collection device of claim 1, wherein the portion of the tissue collection feature that dissolves when placed in the fixation medium comprises polycarbonate.

5. The tissue sample collection device of claim 1, wherein the portion of the tissue collection feature that dissolves when placed in the fixation medium comprises acrylonitrile butadiene styrene (ABS).

6. The tissue sample collection device of claim 1, wherein the portion of the tissue collection feature that dissolves when placed in the fixation medium dissolves in under one hour when placed a fixation medium comprising a 1:1 solution of methanol and acetone.

7. The tissue sample collection device of claim 1, wherein the portion of the tissue collection feature that dissolves when placed in the fixation medium dissolves in under one hour when placed in a fixation medium comprising 100% liquid acetone.

8. The tissue sample collection device of claim 1, wherein the portion of the tissue collection feature that dissolves when placed in the fixation medium dissolves in 100% liquid xylene.

9. The tissue sample collection device of claim 1, wherein the tissue collection feature comprises a brush, the brush comprising a center post and bristles connected to the center post.

10. The tissue sample collection device of claim 9, wherein the portion of the tissue collection feature that dissolves when placed in the fixation medium comprises the bristles.

11. The tissue sample collection device of claim 9, wherein the center post comprises a flat post, and wherein the bristles comprise a micropattern formed on the edges of the center post.

12. A cytology brush comprising:
an elongated shaft including a proximal portion and a distal portion; and
a plurality of bristles disposed on the distal portion,
wherein the bristles are soluble in a fixation medium.

13. The cytology brush of claim 12, wherein the fixation medium comprises one of:
a 1:1 solution of methanol and acetone;
100% liquid acetone;
100% liquid methanol; and
100% liquid ethanol.

14. The cytology brush of claim 12, wherein the bristles comprise a resin.

15. The cytology brush of claim 12, wherein the bristles comprise at least one of:
polycarbonate;
ABS; and
a polycarbonate and ABS blend.

16. The cytology brush of claim 12, wherein the bristles dissolve in under one hour when placed in the fixation medium.

17. The cytology brush of claim 12, wherein the elongated shaft is soluble in the fixation medium.

18. The cytology brush of claim 12, wherein the proximal portion of the elongated shaft comprises a round shaft and wherein the distal portion of the elongated shaft comprises a flat shaft.

19. The cytology brush of claim 12, wherein the elongated shaft comprises a twisted wire.

20. A method of preparing a cytology sample for analysis comprising:
dissolving at least a portion of a cell sample collection device containing cells in a fixation medium.
